SEQUENCE LISTING (24 sequences)

SEQ ID NO: 1 - RNA, 43 nt, Homo sapiens
  source 1..43: mol_type = mRNA
  5'UTR 1..43: note = Native hist1h1c 5 prime UTR
catcggcgctttgccacttgtacccgagtttttgattctcaac

SEQ ID NO: 2 - RNA, 49 nt, synthetic construct
  source 1..49: mol_type = other RNA
  5'UTR 1..49: note = hist1h1c 5 prime UTR + kozak sequence
catcggcgctttgccacttgtacccgagtttttgattctcaacgccacc

SEQ ID NO: 3 - RNA, 49 nt, synthetic construct
  source 1..49: mol_type = other RNA
  misc_feature 1..49: note = Mutant 1
catcgacgctttgccacttgtacccgagtttttgattctcaacgccacc

SEQ ID NO: 4 - RNA, 49 nt, synthetic construct
  source 1..49: mol_type = other RNA
  misc_feature 1..49: note = Mutant 2
catcgacactttgccacttgtacccgagtttttgattctcaacgccacc

SEQ ID NO: 5 - RNA, 49 nt, synthetic construct
  source 1..49: mol_type = other RNA
  misc_feature 1..49: note = Mutant 3
catcgacactttgccacttgtacccgggtttttgattctcaacgccacc

SEQ ID NO: 6 - RNA, 49 nt, synthetic construct
  source 1..49: mol_type = other RNA
  misc_feature 1..49: note = Mutant 4
catcgacactttgccacttgtacccaagtttttgattctcaacgccacc

SEQ ID NO: 7 - RNA, 49 nt, synthetic construct
  source 1..49: mol_type = other RNA
  misc_feature 1..49: note = Mutant 5
catcgacactttgccacttgtacccgaatttttgattctcaacgccacc

SEQ ID NO: 8 - RNA, 49 nt, synthetic construct
  source 1..49: mol_type = other RNA
  misc_feature 1..49: note = Mutant 6
cattcacactttgccacttgtacccgaatttttgactctcaacgccacc

SEQ ID NO: 9 - RNA, 49 nt, synthetic construct
  source 1..49: mol_type = other RNA
  misc_feature 1..49: note = Mutant 7
catcgacactttgccacttgtacccgaatttttgatcctcaacgccacc

SEQ ID NO: 10 - RNA, 49 nt, synthetic construct
  source 1..49: mol_type = other RNA
  misc_feature 1..49: note = Mutant 8
catcgacactttgccacttgtacccgaatttttgattttcaacgccacc

SEQ ID NO: 11 - RNA, 49 nt, synthetic construct
  source 1..49: mol_type = other RNA
  misc_feature 1..49: note = Mutant 9
cattcacactttgccacttgtacccgaattttcgactctcaacgccacc

SEQ ID NO: 12 - RNA, 49 nt, synthetic construct
  source 1..49: mol_type = other RNA
  misc_feature 1..49: note = Mutant 10
cattcacactttgccacttgtacccgaatttccgactctcgccgccacc

SEQ ID NO: 13 - RNA, 49 nt, synthetic construct
  source 1..49: mol_type = other RNA
  misc_feature 1..49: note = Mutant 11
cattcacactttgccacttgtaccgccatttccgactctcgccgccacc

SEQ ID NO: 14 - RNA, 49 nt, synthetic construct
  source 1..49: mol_type = other RNA
  misc_feature 1..49: note = Mutant 12 (UTR3)
cattcacactttgccacttgtaccgccatttccaactctcgccgccacc

SEQ ID NO: 15 - RNA, 50 nt, synthetic construct
  source 1..50: mol_type = other RNA
  misc_feature 1..50: note = Mutant 13 (UTR4)
gcatcgacactttaccacttgtacccaaatttttgactctcaacgccacc

SEQ ID NO: 16 - RNA, 36 nt, Homo sapiens
  source 1..36: mol_type = mRNA
  5'UTR 1..36: note = Native rpl15 5 prime UTR
cctttccgtctggcggcagccatcaggtaagccaag

SEQ ID NO: 17 - RNA, 37 nt, synthetic construct
  source 1..37: mol_type = other RNA
  5'UTR 1..37: note = rpl15 5 prime UTR 5 prime TOP
gcatagaagtctggcggcagccatcaggtaagccaag

SEQ ID NO: 18 - RNA, 37 nt, synthetic construct
  source 1..37: mol_type = other RNA
  5'UTR 1..37: note = rpl15 5 prime UTR + kozak sequence
gcatagaagtctggcggcagccatcaggtaagccacc

SEQ ID NO: 19 - RNA, 37 nt, synthetic construct
  source 1..37: mol_type = other RNA
  misc_feature 1..37: note = Mutant 14
gcatagaagtctgatcgcagccatcaggtaagccacc

SEQ ID NO: 20 - RNA, 37 nt, synthetic construct
  source 1..37: mol_type = other RNA
  misc_feature 1..37: note = Mutant 15
gcatagaagtttgatcgcagccatcggataagccacc

SEQ ID NO: 21 - RNA, 37 nt, synthetic construct
  source 1..37: mol_type = other RNA
  misc_feature 1..37: note = Mutant 16
gcatagaagtttgatcgcagccattgaataagccacc

SEQ ID NO: 22 - RNA, 37 nt, synthetic construct
  source 1..37: mol_type = other RNA
  misc_feature 1..37: note = Mutant 17 (UTR6)
gcatagaagtccgatcgcagccattgaataagccacc

SEQ ID NO: 23 - RNA, 36 nt, synthetic construct
  source 1..36: mol_type = other RNA
  misc_feature 1..36: note = Mutant 18 (UTR7)
catagaagtcgagacacagccactaagtaagccacc

SEQ ID NO: 24 - RNA, 38 nt, synthetic construct
  source 1..38: mol_type = other RNA
  misc_feature 1..38: note = UTR1 (control sequence)
gagagaaaagaagagtaagaagaaatataagagccacc

(19) United States
(12) Patent Application Publication
(10) Pub. No.: US 2025/0019710 A1
(43) Pub. Date: Jan. 16, 2025

(54) MODIFIED 5' UTR

(71) Applicant: NUTCRACKER THERAPEUTICS, INC., Emeryville, CA (US)

(72) Inventors: Samuel Deutch, Emeryville, CA (US); Daniel Omar Frimannsson, Emeryville, CA (US); Ole Haabeth, Emeryville, CA (US)

(21) Appl. No.: 18/713,508

(22) PCT Filed: Nov. 29, 2022
(86) PCT No.: PCT/US2022/051229

§ 371 Date: May 24, 2024

Related U.S. Application Data
(60) Provisional application No. 63/284,261, filed on Nov. 30, 2021.

(51) Int. Cl. C12N 15/67

(52) U.S. Cl. CPC C12N 15/67

(57) ABSTRACT

The disclosure herein provides examples of modified 5′ UTRs and methods of increasing translation of target mRNA using a modified 5′ UTR.

### RELATED APPLICATIONS

The present patent application claims the priority benefit of U.S. Provisional Patent Application Ser. No. 63/284,261, filed Nov. 30, 2021, the content of which is hereby incorporated by reference in its entirety into this disclosure.

### BACKGROUND

To efficiently translate proteins from mRNA, an mRNA molecule comprises several sequences or structural components including a 5′ cap, a 5′ untranslated region (UTR), an open reading frame, a 3′ untranslated region, and a polyadenylated tail. The 5′ and 3′ UTRs are unique regulators for protein translation; specifically, the 5′ UTR modulates the rate of translation while the 3′ UTR affects the rate of transcript degradation. As a regulator of translation rate, de novo discovery and design of 5′ UTRs is an important consideration for the manufacturability and dosing of mRNA therapeutics.

### SUMMARY

In one aspect, the disclosure provides a modified 5′ UTR comprising SEQ ID NO: 14, SEQ ID NO: 15, SEQ ID NO: 22, or SEQ ID NO: 23.

In another aspect, the disclosure provides a polynucleotide sequence comprising a 5′ UTR of hist1h1c or rpl15, wherein the 5′ UTR comprises one or more point mutations that reduce formation of secondary structures in the 5′ UTR.

In some aspects of the polynucleotide sequence, the 5′ UTR is the 5′ UTR of hist1h1c, and the 5′ UTR of hist1h1c lacks a start codon and comprises a kozak consensus sequence at the 3′ terminus of the 5′ UTR of hist1h1c.

In some aspects of the polynucleotide sequence, the 5′ UTR of hist1h1c comprises substitution mutations at positions 6, 8, 26, 28, and 36 from the 5′ terminus of the 5′ UTR of hist1h1c.

In some aspects of the polynucleotide sequence, the 5′ UTR of hist1h1c further comprises substitution mutations at positions 4, 5, 25, 27, 32, 33, 34, 41, and 42 from the 5′ terminus of the 5′ UTR of hist1h1c.

In some aspects of the polynucleotide sequence, the 5′ UTR of hist1h1c further comprises a substitution mutation at position 13 from the 5′ terminus of the 5′ UTR of hist1h1c.

In some aspects of the polynucleotide sequence, the 5′ UTR of hist1h1c further comprises a substitution mutation at position 13 from the 5′ terminus of the 5′ UTR of hist1h1c and an insertion mutation at the 5′ terminus of the 5′ UTR of hist1h1c. In some aspects, the insertion mutation is immediately upstream of position 1 from the 5′ terminus of the 5′ UTR of hist1h1c. In some aspects, the insertion mutation is insertion of a single nucleotide.

In some aspects of the polynucleotide sequence, the 5′ UTR is the 5′ UTR of rpl15, and the 5′ UTR of rpl15 lacks a start codon and comprises a kozak consensus sequence at the 3′ terminus of the 5′ UTR of hist1h1c.

In some aspects of the polynucleotide sequence, the 5′ UTR of rpl15 comprises substitution mutations at positions 11, 13, 14, 15, 24, and 26 from the 5′ terminus of the 5′ UTR of rpl15.

In some aspects of the polynucleotide sequence, the 5′ UTR of rpl15 further comprises substitution mutations at positions 25 and 27 from the 5′ terminus of the 5′ UTR of rpl15.

In some aspects of the polynucleotide sequence, the 5′ UTR of rpl15 further comprises substitution mutations at positions 12, 16, and 23 from the 5′ terminus of the 5′ UTR of rpl15.

In some aspects of the polynucleotide sequence, the 5′ UTR of rpl15 further comprises substitution mutations at positions 25 and 27 from the 5′ terminus of the 5′ UTR of rpl15 and an insertion mutation at the 5′ terminus of the 5′ UTR of rpl15. In some aspects, the insertion mutation is immediately upstream of position 1 from the 5′ terminus of the 5′ UTR of rpl15. In some aspects, the insertion mutation is insertion of a single nucleotide.

In some aspects of the polynucleotide sequence, the polynucleotide sequence further comprises a coding region located downstream of the 5′ UTR of hist1h1c or rpl15. In some aspects, the polynucleotide sequence includes a 5′ UTR with a sequence selected from SEQ ID NO: 14, SEQ ID NO: 15, SEQ ID NO: 22, or SEQ ID NO: 23.

In another aspect, the disclosure provides a method of increasing translation of target mRNA, wherein the method comprises promoting translation with a modified 5′ UTR comprising SEQ ID NO: 14, SEQ ID NO: 15, SEQ ID NO: 22, or SEQ ID NO: 23, wherein the modified 5′ UTR is upstream of the target mRNA in a polynucleotide sequence.

In yet another aspect, the disclosure provides a method of increasing translation of target mRNA, wherein the method comprises promoting translation with a modified 5′ UTR of hist1h1c or rpl15, wherein the 5′ UTR comprises one or more point mutations that reduce formation of secondary structures in the 5′ UTR, wherein the modified 5′ UTR is upstream of the target mRNA in a nucleotide sequence.

In some aspects of the method, the 5′ UTR is the 5′ UTR of hist1h1c, and the 5′ UTR of hist1h1c lacks a start codon and comprises a kozak consensus sequence at the 3′ terminus of the 5′ UTR of hist1h1c.

In some aspects of the method, the 5′ UTR of hist1h1c comprises substitution mutations at positions 6, 8, 26, 28, and 36 from the 5′ terminus of the 5′ UTR of hist1h1c.

In some aspects of the method, the 5′ UTR of hist1h1c further comprises substitution mutations at positions 4, 5, 25, 27, 32, 33, 34, 41, and 42 from the 5′ terminus of the 5′ UTR of hist1h1c.

In some aspects of the method, the 5′ UTR of hist Ihle further comprises a substitution mutation at position 13 from the 5′ terminus of the 5′ UTR of hist1h1c.

In some aspects of the method, the 5′ UTR of hist Ihle further comprises a substitution mutation at position 13 from the 5′ terminus of the 5′ UTR of hist1h1c and an insertion mutation at the 5′ terminus of the 5′ UTR of hist1h1c. In some aspects, the insertion mutation is immediately upstream of position 1 from the 5′ terminus of the 5′ UTR of hist1h1c. In some aspects, the insertion mutation is insertion of a single nucleotide.

In some aspects of the method, the 5′ UTR is the 5′ UTR of rpl15, and the 5′ UTR of rpl15 lacks a start codon and comprises a kozak consensus sequence at the 3′ terminus of the 5′ UTR of hist1h1c.

In some aspects of the method, the 5′ UTR of rpl15 comprises substitution mutations at positions 11, 13, 14, 15, 24, and 26 from the 5′ terminus of the 5′ UTR of rpl15.

In some aspects of the method, the 5′ UTR of rpl15 further comprises substitution mutations at positions 25 and 27 from the 5′ terminus of the 5′ UTR of rpl15.

In some aspects of the method, the 5′ UTR of rpl15 further comprises substitution mutations at positions 12, 16, and 23 from the 5′ terminus of the 5′ UTR of rpl15.

In some aspects of the method, the 5′ UTR of rpl15 further comprises substitution mutations at positions 25 and 27 from the 5′ terminus of the 5′ UTR of rpl15 and an insertion mutation at the 5′ terminus of the 5′ UTR of rpl15. In some aspects, the insertion mutation is immediately upstream of position 1 from the 5′ terminus of the 5′ UTR of rpl15. In some aspects, the insertion mutation is insertion of a single nucleotide.

Other aspects and features of the present disclosure will become apparent to those ordinarily skilled in the art upon review of the following description of specific aspects of the disclosure in conjunction with the accompanying figures.

It should be appreciated that all combinations of the foregoing concepts and additional concepts discussed in greater detail below (provided such concepts are not mutually inconsistent) are contemplated as being part of the inventive subject matter disclosed herein and may be employed to achieve the benefits as described herein.

### BRIEF DESCRIPTION OF THE DRAWINGS

FIG. 1A illustrates, in one implementation, the resulting secondary RNA structure of the hist1h1c 5′ UTR with a kozak consensus sequence (SEQ ID NO: 2).

FIG. 1B illustrates, in one implementation, the resulting secondary RNA structure of the hist1h1c 5′ UTR with a kozak consensus sequence and the point mutations according to Mutant 1 (SEQ ID NO: 3).

FIG. 1C illustrates, in one implementation, the resulting secondary RNA structure of the hist1h1c 5′ UTR with a kozak consensus sequence and the point mutations according to Mutant 2 (SEQ ID NO: 4).

FIG. 1D illustrates, in one implementation, the resulting secondary RNA structure of the hist1h1c 5′ UTR with a kozak consensus sequence and the point mutations according to Mutant 3 (SEQ ID NO: 5).

FIG. 1E illustrates, in one implementation, the resulting secondary RNA structure of the hist1h1c 5′ UTR with a kozak consensus sequence and the point mutations according to Mutant 4 (SEQ ID NO: 6).

FIG. 1F illustrates, in one implementation, the resulting secondary RNA structure of the hist1h1c 5′ UTR with a kozak consensus sequence and the point mutations according to Mutant 5 (SEQ ID NO: 7).

FIG. 1G illustrates, in one implementation, the resulting secondary RNA structure of the hist1h1c 5′ UTR with a kozak consensus sequence and the point mutations according to Mutant 6 (SEQ ID NO: 8).

FIG. 1H illustrates, in one implementation, the resulting secondary RNA structure of the hist1h1c 5′ UTR with a kozak consensus sequence and the point mutations according to Mutant 7 (SEQ ID NO: 9).

FIG. 1I illustrates, in one implementation, the resulting secondary RNA structure of the hist1h1c 5′ UTR with a kozak consensus sequence and the point mutations according to Mutant 8 (SEQ ID NO: 10).

FIG. 1J illustrates, in one implementation, the resulting secondary RNA structure of the hist1h1c 5′ UTR with a kozak consensus sequence and the point mutations according to Mutant 10 (SEQ ID NO: 12).

FIG. 1K illustrates, in one implementation, the resulting secondary RNA structure of the hist1h1c 5′ UTR with a kozak consensus sequence and the point mutations according to Mutant 11 (SEQ ID NO: 13).

FIG. 1L illustrates, in one implementation, the resulting secondary RNA structure of the hist1h1c 5′ UTR with a kozak consensus sequence and the point mutations according to Mutant 12 (SEQ ID NO: 14).

FIG. 1M illustrates, in one implementation, the resulting secondary RNA structure of the hist1h1c 5′ UTR with a kozak consensus sequence and the point mutations according to Mutant 13 (SEQ ID NO: 15).

FIG. 2A illustrates, in one implementation, the resulting secondary RNA structure of the rpl15 5′ UTR with a kozak consensus sequence (SEQ ID NO: 18).

FIG. 2B illustrates, in one implementation, the resulting secondary RNA structure of the rpl15 5′ UTR with a kozak consensus sequence and the point mutations according to Mutant 14 (SEQ ID NO: 19).

FIG. 2C illustrates, in one implementation, the resulting secondary RNA structure of the rpl15 5′ UTR with a kozak consensus sequence and the point mutations according to Mutant 15 (SEQ ID NO: 20).

FIG. 2D illustrates, in one implementation, the resulting secondary RNA structure of the rpl15 5′ UTR with a kozak consensus sequence and the point mutations according to Mutant 16 (SEQ ID NO: 21).

FIG. 2E illustrates, in one implementation, the resulting secondary RNA structure of the rpl15 5′ UTR with a kozak consensus sequence and the point mutations according to Mutant 17 (SEQ ID NO: 22).

FIG. 2F illustrates, in one implementation, the resulting secondary RNA structure of the rpl15 5′ UTR with a kozak consensus sequence and the point mutations according to Mutant 18 (SEQ ID NO: 23).

FIG. 3 is a graph depicting, in one implementation, a time course of luciferase expression in HEK293 cells transfected with an expression vector comprising a modified 5′ UTR of the disclosure.

FIG. 4 is a graph depicting, in one implementation, luciferase expression at 12 hours post-transfection in JAWSII cells transfected with an expression vector comprising a modified 5′ UTR of the disclosure.

FIG. 5 is a graph depicting, in one implementation, a time course of luciferase expression in PBMCs transfected with an expression vector comprising a modified 5′ UTR of the disclosure.

### DETAILED DESCRIPTION

### Terminology

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

Identity: As used herein, the term “identity” refers to the overall monomer conservation between polymeric molecules, e.g., between polynucleotide molecules (e.g., DNA molecules and/or RNA molecules) and/or between polypeptide molecules. Calculation of the percent identity of two polynucleotide sequences, for example, can be performed by aligning the two sequences for optimal comparison purposes (e.g., gaps can be introduced in one or both of a first and a second nucleic acid sequence for optimal alignment and non-identical sequences can be disregarded for comparison purposes). In certain aspects, the length of a sequence aligned for comparison purposes is at least about 30%, at least about 40%, at least about 50%, at least about 60%, at least about 70%, at least about 80%, at least about 90%, at least about 95%, or at least about 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. When comparing DNA and RNA, thymine (T) and uracil (U) can be considered equivalent.

Suitable software programs are available from various sources and for alignment of both protein and nucleotide sequences. One suitable program to determine percent sequence identity is b12seq, part of the BLAST suite of programs available from the U.S. government's National Center for Biotechnology Information BLAST website (blast.ncbi.nlm.nih.gov). B12seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI).

Sequence alignments can be conducted using methods such as, but not limited to, MAFFT, Clustal (ClustalW, Clustal X or Clustal Omega), or MUSCLE.

Different regions within a single polynucleotide or polypeptide target sequence that aligns with a polynucleotide or polypeptide reference sequence can each have their own percent sequence identity.

In Vitro: As used herein, the term “in vitro” refers to events that occur in an artificial environment, e.g., in a test tube or reaction vessel, in cell culture, in a Petri dish, etc., rather than within an organism (e.g., animal, plant, or microbe).

In Vivo: As used herein, the term “in vivo” refers to events that occur within an organism (e.g., animal, plant, or microbe or cell or tissue thereof).

Isolated: As used herein, the term “isolated” refers to a substance or entity that has been separated from at least some of the components with which it was associated (whether in nature or in an experimental setting). Isolated substances (e.g., nucleotide sequence or protein sequence) can have varying levels of purity in reference to the substances from which they have been associated. Isolated substances and/or entities can be separated from at least about 10%, about 20%, about 30%, about 40%, about 50%, about 60%, about 70%, about 80%, about 90%, or more of the other components with which they were initially associated. In some aspects, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. The term “substantially isolated” means that the compound is substantially separated from the environment in which it was formed or detected. Partial separation can include, for example, a composition enriched in the compound of the present disclosure. Substantial separation can include compositions containing at least about 50%, at least about 60%, at least about 70%, at least about 80%, at least about 90%, at least about 95%, at least about 97%, or at least about 99% by weight of the compound of the present disclosure, or salt thereof.

A polynucleotide, vector, polypeptide, cell, or any composition disclosed herein which is “isolated” is a polynucleotide, vector, polypeptide, cell, or composition which is in a form not found in nature. Isolated polynucleotides, vectors, polypeptides, or compositions include those that have been purified to the degree that they are no longer in a form in which they are found in nature. In some aspects, a polynucleotide, vector, polypeptide, or composition that is isolated is substantially pure.

Nucleotides are referred to by their commonly accepted single-letter codes. Unless otherwise indicated, nucleic acids are written left to right in 5′ to 3′ orientation. Nucleotides are referred to herein by their commonly known one-letter symbols recommended by the IUPAC-IUB Biochemical Nomenclature Commission. Accordingly, A represents adenine, C represents cytosine, G represents guanine, T represents thymine, and U represents uracil.

Messenger RNA (mRNA): As used herein, the term “messenger RNA” (mRNA) refers to any polynucleotide that encodes a polypeptide of interest and is capable of being translated to produce the encoded polypeptide in vitro, in vivo, in situ, or ex vivo.

Native or naturally occurring: As used herein, a “native” or “naturally occurring” polynucleotide sequence means a polynucleotide sequence existing in nature without artificial aid.

Nucleic acid sequence: The terms “nucleic acid sequence,” “nucleotide sequence,” or “polynucleotide sequence” are used interchangeably and refer to a continuous nucleic acid sequence. The sequence can be either single stranded or double stranded DNA or RNA, e.g., an mRNA.

The term “nucleic acid,” in its broadest sense, includes any compound and/or substance that comprises a polymer of nucleotides. These polymers are often referred to as polynucleotides. Example nucleic acids or polynucleotides of the disclosure include, but are not limited to, ribonucleic acids (RNAs) or deoxyribonucleic acids (DNAs).

The phrase “nucleotide sequence encoding” refers to the nucleic acid (e.g., an mRNA or DNA molecule) coding sequence that encodes a polypeptide. As used herein, the terms “coding region” and “coding sequence”, refer to an Open Reading Frame (ORF) in a polynucleotide that upon expression, yields a polypeptide or protein. The coding sequence can further include initiation and termination signals operably linked to regulatory elements, including a promoter and polyadenylation signal capable of directing expression in the cells of an individual or mammal to which the nucleic acid is administered. The coding sequence can further include sequences that encode signal peptides.

Open reading frame: As used herein, “open reading frame” or “ORF” refers to a sequence that does not contain a stop codon in a given reading frame.

Part: As used herein, a “part” or “region” of a polynucleotide is defined as any portion of the polynucleotide that is less than the entire length of the polynucleotide. Likewise, a “part” or “region” of a polypeptide is defined as any portion of the polypeptide that is less than the entire length of the polynucleotide.

Point mutation: As used herein, “point mutation” refers to a genetic mutation in which a single nucleobase is substituted, inserted, or deleted from a polynucleotide sequence. The term “nucleobase substitution”, “substitution”, or “substitution mutation” as used herein refers to replacing a single nucleobase present in a reference polynucleotide sequence (e.g., a wild type or native sequence) with another nucleobase. Accordingly, a reference to a “substitution at position X” refers to the substitution of a nucleobase present at position X with an alternative nucleobase.

As used herein, “nucleobase insertion”, “insertion”, or “insertion mutation” refers to inserting a single nucleobase immediately adjacent to a nucleobase at a particular position of a reference polynucleotide sequence. As used herein, “nucleobase deletion”, “deletion”, or “deletion mutation” refers to deleting a single nucleobase immediately adjacent to a nucleobase at a particular position of a reference polynucleotide sequence.

Polynucleotide: The term “polynucleotide” as used herein refers to polymers of nucleotides of any length, including ribonucleotides, deoxyribonucleotides, analogs thereof, or mixtures thereof. This term refers to the primary structure of the molecule. Thus, the term includes triple-, double- and single-stranded deoxyribonucleic acid (“DNA”), as well as triple-, double- and single-stranded ribonucleic acid (“RNA”). More particularly, the term “polynucleotide” includes polydeoxyribonucleotides (containing 2-deoxy-D-ribose), polyribonucleotides (containing D-ribose), including tRNA, IRNA, hRNA, siRNA, and mRNA, whether spliced or unspliced, any other type of polynucleotide which is an N- or C-glycoside of a purine or pyrimidine base, and other polymers containing normucleotidic backbones, for example, polyamide (e.g., peptide nucleic acids “PNAs”) and polymorpholino polymers, and other synthetic sequence-specific nucleic acid polymers providing that the polymers contain nucleobases in a configuration which allows for base pairing and base stacking, such as is found in DNA and RNA. In particular aspects, the polynucleotide comprises an mRNA.

The T bases in the codon maps disclosed herein are present in DNA, whereas the T bases may be replaced by U bases in corresponding RNAs. For example, a codon-nucleotide sequence disclosed herein in DNA form, e.g., a vector or an in vitro translation (IVT) template, may have its T bases transcribed as U based in its corresponding transcribed mRNA. In this respect, both codon-optimized DNA sequences (comprising T) and their corresponding RNA sequences (comprising U) are considered codon-optimized nucleotide sequences of the present disclosure. Equivalent codon-maps can be generated by replacing one or more bases with non-natural bases. Thus, e.g., a TTC codon (DNA map) may correspond to a UUC codon (RNA map), which in turn may correspond to a ‘P’C codon (RNA map in which U has been replaced with pseudouridine).

Standard A-T and G-C base pairs form under conditions that allow the formation of hydrogen bonds between the N3-H and C4-oxy of thymidine and the NI and C6-NH₂, respectively, of adenosine and between the C2-oxy, N3, and C4-NH₂, of cytidine and the C2-NH₂, N′—H and C6-oxy, respectively, of guanosine. Thus, for example, guanosine (2-amino-6-oxy-9-β-D-ribofuranosyl-purine) can be modified to form isoguanosine (2-oxy-6-amino-9-β-D-ribofuranosyl-purine). Such modification results in a nucleoside base, which will no longer effectively form a standard base pair with cytosine. However, modification of cytosine (1-β-D-ribofuranosyl-2-oxy-4-amino-pyrimidine) to form isocytosine (1-β-D-ribofuranosyl-2-amino-4-oxy-pyrimidine-) results in a modified nucleotide which will not effectively base pair with guanosine but will form a base pair with isoguanosine.

Polypeptide: The terms “polypeptide,” “peptide,” and “protein” are used interchangeably herein to refer to polymers of amino acids of any length. The polymer can comprise modified amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids such as homocysteine, ornithine, p-acetylphenylalanine, D-amino acids, and creatine).

The term, as used herein, refers to proteins, polypeptides, and peptides of any size, structure, or function. Polypeptides include gene products, naturally occurring polypeptides, synthetic polypeptides, homologs, orthologs, paralogs, fragments and other equivalents, variants, and analogs of the foregoing. A polypeptide can be a single polypeptide or can be a multi-molecular complex such as a dimer, trimer, or tetramer. They can also comprise single chain or multichain polypeptides. Most commonly, disulfide linkages are found in multichain polypeptides. The term polypeptide can also apply to amino acid polymers in which one or more amino acid residues are an artificial chemical analogue of a corresponding naturally occurring amino acid. In some aspects, a “peptide” can be less than or equal to about 50 amino acids long, e.g., about 5, 10, 15, 20, 25, 30, 35, 40, 45, or 50 amino acids long.

Reference Nucleic Acid Sequence: The term “reference nucleic acid sequence”, “reference nucleic acid”, or “reference nucleotide sequence” or “reference sequence” refers to a starting nucleic acid sequence (e.g., a RNA, e.g., an mRNA sequence) that can be sequence optimized. In some aspects, the reference nucleic acid sequence is a wild type or native nucleic acid sequence, a fragment or a variant thereof.

Sequence Optimization: As used herein, “sequence optimization” refers to a process or series of processes by which nucleobases in a reference nucleic acid sequence are replaced with alternative nucleobases, resulting in a nucleic acid sequence with improved properties. In the context of the present disclosure, sequence optimization refers to modifications in a nucleotide sequence of a 5′ UTR that result in improved translation of a downstream gene target when the 5′ UTR is incorporated into a suitable expression system.

Similarity: As used herein, the term “similarity” refers to the overall relatedness between polymeric molecules, e.g., between polynucleotide molecules (e.g., DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions.

Substantially: As used herein, the term “substantially” refers to the qualitative condition of exhibiting total or near-total extent or degree of a characteristic or property of interest. Biological and chemical phenomena rarely, if ever, go to completion and/or proceed to completeness or achieve or avoid an absolute result. The term “substantially” is therefore used herein to capture the potential lack of completeness inherent in many biological and chemical phenomena. For example, “substantially” may refer to being within at least about 20%, alternatively at least about 10%, alternatively at least about 5% of a characteristic or property of interest.

Synthetic: The term “synthetic” means produced, prepared, and/or manufactured by the hand of man. Synthesis of polynucleotides or other molecules of the present disclosure can be chemical or enzymatic.

Terminus: As used herein, the terms “termini” or “terminus,”, when referring to polypeptides, refers to an extremity of a peptide or polypeptide. Such extremity is not limited only to the first or final site of the peptide or polypeptide but can include additional amino acids in the terminal regions. The polypeptide based molecules of the disclosure can be characterized as having both an N-terminus (terminated by an amino acid with a free amino group (NH₂)) and a C-terminus (terminated by an amino acid with a free carboxyl group (COOH)). Proteins of the disclosure are in some cases made up of multiple polypeptide chains brought together by disulfide bonds or by non-covalent forces (multimers, oligomers). These sorts of proteins will have multiple N- and C-termini. Alternatively, the termini of the polypeptides can be modified such that they begin or end, as the case can be, with a non-polypeptide-based moiety such as an organic conjugate.

Transfection: As used herein, “transfection” refers to the introduction of a polynucleotide into a cell wherein a polypeptide encoded by the polynucleotide is expressed (e.g., mRNA) or the polypeptide modulates a cellular function (e.g., siRNA, miRNA). As used herein, “expression” of a nucleic acid sequence refers to the translation of a polynucleotide (e.g., an mRNA) into a polypeptide or protein and/or post-translational modification of a polypeptide or protein.

Unmodified: As used herein, “unmodified” refers to any substance, compound or molecule prior to being changed in any way. Unmodified can, but does not always, refer to the wild type or native form of a biomolecule. Molecules can undergo a series of modifications whereby each modified molecule can serve as the “unmodified” starting molecule for a subsequent modification.

Untranslated region: As used herein “untranslated region” or “UTR” refer to regions located at the 5′ and 3′ ends of an mRNA construct that do not form a protein-coding region. The 5′ UTR is upstream from a coding sequence.

### Modified 5′ UTRs

Described herein are example polynucleotide sequences, and more specifically, 5′ UTRs derived from the 5′ UTR of hist1h1c (i.e., “modified hist1h1c 5′ UTR”) or rpl15 5′ UTR (i.e., “modified rpl15 5′ UTR”). A modified 5′ UTRs can be used to promote expression of a target polynucleotide (e.g., target mRNA) when incorporated into an expression construct or vector. As used herein, a “modified” 5′ UTR refers to a 5′ UTR sequence resulting from sequence optimization.

In one aspect, polynucleotide sequences that constitute a modified 5′ UTR of the disclosure share the following characteristics:
a. derived from the native 5′ UTR of hist1h1c or rpl15;
b. the lack of one or more stop codons;
c. the lack of a 5′ terminal oligopyrimidine tract (5′ TOP) inhibitory motif;
d. the presence of a kozak consensus sequence at the 3′ terminus; and
e. the presence of one or more point mutations that inhibit, or at least reduce, the formation of secondary structure in the 5′ UTR.

In some aspects, the modified 5′ UTRs include the polynucleotide sequences shown in Table 1.

TABLE 1
Sequences of Modified 5′ UTRs

| | | Sequence |
|---|---|---|
| Sequence | SEQ ID NO | Identifier |
| CATTCACACTTTGCCACTTGTACCGCCATTTCCAACTCTCGCCGCCACC | SEQ ID NO: 14 | UTR3 |
| GCATCGACACTTTACCACTTGTACCCAAATTTTTGACTCTCAACGCCACC | SEQ ID NO: 15 | UTR4 |
| GCATAGAAGTCCGATCGCAGCCATTGAATAAGCCACC | SEQ ID NO: 22 | UTR6 |
| CATAGAAGTCGAGACACAGCCACTAAGTAAGCCACC | SEQ ID NO: 23 | UTR7 |
| GAGAGAAAAGAAGAGTAAGAAGAAATATAAGAGCCACC | SEQ ID NO: 24 | UTR1 |

In example aspects, the modified 5′ UTR comprises SEQ ID NO: 15 or SEQ ID NO: 23.

In some aspects, the modified 5′ UTR comprises a combination of SEQ ID NO: 14, SEQ ID NO: 15, SEQ ID NO: 22, and/or SEQ ID NO: 23. In some aspects, the modified 5′ UTR comprises a combination of SEQ ID NO: 15 and SEQ ID NO: 23. In some aspects, the modified 5′ UTR comprises SEQ ID NO: 23.

In some aspects, the 5′ UTR is a modified hist1h1c 5′ UTR that comprises one or more point mutations that reduce formation of secondary structure in the 5′ UTR.

In some aspects, the modified hist1h1c 5′ UTR lacks all start codons.

In some aspects, the modified hist1h1c 5′ UTR lacks a 5′ TOP inhibitory motif.

In some aspects, the modified hist1h1c 5′ UTR has a kozak consensus sequence at the 3′ terminus.

In some aspects, the modified hist1h1c 5′ UTR comprises a substitution mutation at positions 6, 8, 26, 28, and 36 from the 5′ terminus of the 5′ UTR.

In some aspects, the modified hist1h1c 5′ UTR comprises a substitution mutation at positions 4, 5, 6, 8, 25, 26, 27, 28, 32, 33, 34, 36, 41, and 42 from the 5′ terminus of the 5′ UTR.

In some aspects, the modified hist1h1c 5′ UTR comprises a substitution mutation at positions 6, 8, 13, 26, 28, and 36 from the 5′ terminus of the 5′ UTR.

In some aspects, the modified hist1h1c 5′ UTR comprises an insertion mutation at the 5′ terminus of the 5′ UTR, wherein the insertion is immediately upstream (i.e., the preceding nucleotide) of position 1 from the 5′ terminus of the 5′ UTR.

In some aspects, the polynucleotide sequence that constitutes a modified hist1h1c 5′ UTR is characterized by the following:
a. the lack of a start codon;
b. the lack of a 5′ TOP inhibitory motif;
c. the presence of a kozak consensus sequence at the 3′ terminus; and
d. the presence of a substitution mutation at positions 6, 8, 26, 28, and 36 from the 5′ terminus of the 5′ UTR.

In some aspects, the polynucleotide sequence that constitutes a modified hist1h1c 5′ UTR is characterized by the following:
a. the lack of a start codon;
b. the lack of a 5′ TOP inhibitory motif;
c. the presence of a kozak consensus sequence at the 3′ terminus; and
d. the presence of a substitution mutation at positions 4, 5, 6, 8, 25, 26, 27, 28, 32, 33, 34, 36, 41, and 42 from the 5′ terminus of the 5′ UTR.

In some aspects, the polynucleotide sequence that constitutes a modified hist1h1c 5′ UTR is characterized by the following:
a. the lack of a start codon;
b. the lack of a 5′ TOP inhibitory motif;
c. the presence of a kozak consensus sequence at the 3′ terminus; and
d. the presence of a substitution mutation at positions 6, 8, 13, 26, 28, and 36 from the 5′ terminus of the 5′ UTR.

In some aspects, the polynucleotide sequence that constitutes a modified hist1h1c 5′ UTR is characterized by the following:
a. the lack of a start codon;
b. the lack of a 5′ TOP inhibitory motif;
c. the presence of a kozak consensus sequence at the 3′ terminus;
d. the presence of a substitution mutation at positions 6, 8, 13, 26, 28, and 36 from the 5′ terminus of the 5′ UTR; and
e. the presence of an insertion mutation immediately upstream of position 1 from the 5′ terminus of the 5′ UTR.

In some aspects, the 5′ UTR is a modified rpl15 5′ UTR that comprises one or more point mutations that reduce formation of secondary structure in the 5′ UTR.

In some aspects, the modified rpl15 5′ UTR lacks all start codons.

In some aspects, the modified rpl15′ UTR lacks a 5′ TOP inhibitory motif.

In some aspects, the modified rpl15′ UTR has a kozak consensus sequence at the 3′ terminus.

In some aspects, the modified rpl15 5′ UTR comprises a substitution mutation at positions 11, 13, 14, 15, 24, and 26 from the 5′ terminus of the 5′ UTR.

In some aspects, the modified rpl15 5′ UTR comprises a substitution mutation at positions 11, 13, 14, 15, 24, 25, 26, and 27 from the 5′ terminus of the 5′ UTR.

In some aspects, the modified rpl15 5′ UTR comprises a substitution mutation at positions 11, 12, 13, 14, 15, 16, 23, 24, and 26 from the 5′ terminus of the 5′ UTR.

In some aspects, the modified rpl15 5′ UTR comprises an insertion mutation at the 5′ terminus of the 5′ UTR, wherein the insertion is immediately upstream (i.e., the preceding nucleotide) of position 1 from the 5′ terminus of the 5′ UTR.

In some aspects, the polynucleotide sequence that constitutes a modified rpl15 5′ UTR is characterized by the following:
a. the lack of a start codon;
b. the lack of a 5′ TOP inhibitory motif;
c. the presence of a kozak consensus sequence at the 3′ terminus; and
d. the presence of a substitution mutation at positions 11, 13, 14, 15, 24, and 26 from the 5′ terminus of the 5′ UTR.

In some aspects, the polynucleotide sequence that constitutes a modified rpl15 5′ UTR is characterized by the following:
a. the lack of a start codon;
b. the lack of a 5′ TOP inhibitory motif;
c. the presence of a kozak consensus sequence at the 3′ terminus; and
d. the presence of a substitution mutation at positions 11, 13, 14, 15, 24, 25, 26, and 27 from the 5′ terminus of the 5′ UTR.

In some aspects, the polynucleotide sequence that constitutes a modified rpl15 5′ UTR is characterized by the following:
a the lack of a start codon;
b. the lack of a 5′ TOP inhibitory motif;
c. the presence of a kozak consensus sequence at the 3′ terminus; and
d. the presence of a substitution mutation at positions 11, 12, 13, 14, 15, 16, 23, 24, and 26 from the 5′ terminus of the 5′ UTR.

In some aspects, the polynucleotide sequence that constitutes a modified rpl15 5′ UTR is characterized by the following:
a. the lack of a start codon;
b. the lack of a 5′ TOP inhibitory motif;
c. the presence of a kozak consensus sequence at the 3′ terminus;
d. the presence of a substitution mutation at positions 11, 13, 14, 15, 24, 25, 26, and 27 from the 5′ terminus of the 5′ UTR; and
e. the presence of an insertion mutation immediately upstream of position 1 from the 5′ terminus of the 5′ UTR.

### Methods of Increasing Translation Efficiency

In another aspect, the disclosure provides methods of expressing a target polynucleotide (e.g., mRNA) using a modified 5′ UTR as described herein. In some aspects, a modified 5′ UTR and target polynucleotide can be operably linked to one or more regulatory nucleotide sequences in an expression construct. In some aspects, the nucleic acid sequences of the 5′ UTR and target polynucleotide can be expressed using a vector having regulatory nucleotide sequences. Regulatory nucleotide sequences and can include promoter sequences, leader or signal sequences, ribosomal binding sites, transcriptional start and termination sequences, translational start and termination sequences, and enhancer sequences. Promoters herein may refer to naturally occurring promoters and hybrid promoters that combine elements of more than one promoter. Any suitable expression vectors and suitable regulatory sequences can be selected and optimized for a particular host cell. Any suitable constitutive or inducible promoter is contemplated for use with the modified 5′ UTRs of the disclosure. Expression constructs can be present in a cell on an episome, such as a plasmid, or inserted into a chromosome.

In some aspects, an expression vector can contain a selectable marker gene for selection of transformed host cells. Selectable marker genes can be selected for a particular host cell.

A host cell can be transfected with an expression vector comprising a modified 5′ UTR and a target polynucleotide, and can be cultured under conditions suitable for expression of the target polynucleotide product (e.g., protein). In one implementation, target protein can be isolated from cells and/or cell culture medium using suitable purification techniques such as ion-exchange chromatography, gel filtration chromatography, ultrafiltration, electrophoresis, and immunoaffinity purification.

Suitable expression vectors include plasmids such as pBR322-derived plasmids, pEMBL-derived plasmids, pEX-derived plasmids, pBTac-derived plasmids, and pUC-derived plasmids for expression in prokaryotic cells, such as E. coli. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of proteins in eukaryotic cells.

Making recombinant polynucleotides may involve joining various nucleic acid fragments coding for different polypeptide sequences or translational regulatory sequences.

In some aspects, an expression vector comprising a modified 5′ UTR of the disclosure can be used to express a target polynucleotide in a host cell such as bacterial cells (e.g., E. coli), insect cells (e.g., using a baculovirus expression system), yeast, or mammalian cells. Other suitable host cells may be employed. Thus, the disclosure includes polynucleotides comprising a modified 5′ UTR and a target polynucleotide and host cells expressing the polynucleotide.

In example aspects, an expression construct comprises a modified 5′ UTR of the disclosure, an effector region encoding a protein (e.g., mRNA), and a 3′ UTR.

In a further aspect, the disclosure provides methods of increasing translation of a target polynucleotide using a modified 5′ UTR as described herein. The method comprises promoting translation or a target polynucleotide with a modified 5′ UTR, wherein the 5′ UTR is upstream of the target polynucleotide in a polynucleotide sequence. The polynucleotides comprising a modified 5′ UTR and a target polynucleotide and/or the host cells as described herein can be utilized in a method of increasing translation of target polynucleotide.

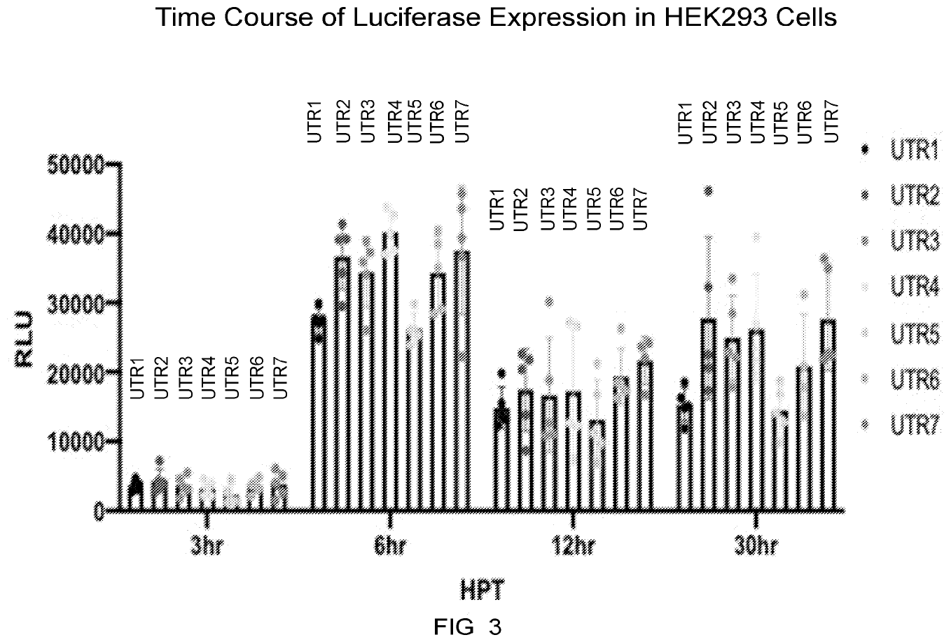

Example 3 and FIGS. 3-5 are non-limiting examples of increasing translation of a target polynucleotide using a modified 5′ UTR according to an aspect of the disclosure.

### Compositions

In yet another aspect, the disclosure provides compositions comprising polynucleotides or host cells expressing polynucleotides as described herein (i.e., polynucleotides comprising a modified 5′ UTR and a target polynucleotide).

In some aspects, the composition is a therapeutic composition that includes one or more pharmaceutically acceptable carriers, diluents, or excipients such as salts, buffering agents, preservatives, antiadherents, antioxidants, binders, coatings, compression aids, disintegrants, dyes, emollients, emulsifiers, fillers, film formers or coatings, flavors, fragrances, glidants, lubricants, sorbents, suspending or dispersing agents, sweeteners, waters of hydration, and/or other therapeutic agents.

In some aspects, the composition comprises an expression construct comprising a modified 5′ UTR of the disclosure, an effector region encoding a protein (e.g., mRNA), and a 3′ UTR.

In some aspects, a therapeutic composition can be used to treat a disease or disorder in a subject. In some aspects, a therapeutic composition can used to prevent a disease or disorder in a subject.

### Kits

Another aspect of the present disclosure is a kit comprising a modified 5′ UTR, composition, polynucleotide, expression construct, or host cell as described herein, and instructions or a label directing appropriate use or administration. Typically, kits will comprise sufficient amounts and/or numbers of components to allow a user to perform one or multiple treatments of a subject(s) and/or to perform one or multiple experiments.

In some aspects, additional components for conducting research assays and/or for administering therapeutically effective amounts of a polynucleotide can be enclosed in the kit.

In some aspects, the kit can include instructions for cloning a modified 5′ UTR into an expression construct to encode a target polypeptide.

In some aspects, the kit comprises an expression construct comprising a modified 5′ UTR of the disclosure, an effector region encoding a protein (e.g., mRNA), and a 3′ UTR.

In some aspects, the kit can include instructions for making a polypeptide by, for example, culturing a host cell as described herein.

### EQUIVALENTS AND SCOPE

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes aspects in which exactly one member of the group is present in, employed in, or otherwise relevant to a given product or process. The disclosure includes aspects in which more than one, or the entire group members are present in, employed in, or otherwise relevant to a given product or process.

As used herein, “about” as used in connection with a numerical value throughout the specification and/or the claims denotes an interval of accuracy. In general, such interval of accuracy is ±10%. “Approximately” as applied to one or more values of interest, refers to a value that is similar to a stated reference value. In certain aspects, the term “approximately” refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context (except where such number may exceed 100% of a possible value).

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context, values that are expressed as ranges can assume any specific value or subrange within the stated ranges in different aspects of the disclosure, to the tenth of the unit of the lower limit of the range, unless the context clearly dictates otherwise.

It is to be understood that the words which have been used are words of description rather than limitation and that changes may be made within the purview of the appended claims without departing from the true scope and spirit of the disclosure in its broader aspects.

While the present disclosure has been described at some length and with some particularity with respect to the several described aspects, it is not intended that it should be limited to any such particulars or aspects or any particular aspect, but it is to be construed with references to the appended claims so as to provide the broadest possible interpretation of such claims in view of the prior art and, therefore, to effectively encompass the intended scope of the disclosure.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Wherever aspects are described herein with the language “comprising,” otherwise analogous aspects described in terms of “consisting of” and/or “consisting essentially of” are also provided.

Units, prefixes, and symbols are denoted in their Systeme International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither, or both limits are included is also encompassed within the disclosure. Where a value is explicitly recited, it is to be understood that values which are about the same quantity or amount as the recited value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

The present disclosure is further illustrated by the following non-limiting examples.

### EXAMPLES

### Example 1: 5′ UTR Discovery

To identify candidate 5′ UTRs, JMP statistical software was utilized to search for genes having a high translation rate. This initial search criterion identified approximately 5000 candidate genes. These candidates were subsequently sorted by the size of their respective 5′ UTRs using UTRdb (ITB—INSTITUTE FOR BIOMEDICAL TECHNOLOGIES). Genes having a 5′ UTR of greater than 60 nucleobases were removed from further investigation as 5′ UTRs of less than 60 nucleobases allowed for rapid DNA template synthesis using PCR. This resulted in 50 candidates, which were subsequently screened for cellular expression using The Human Protein Atlas. From these 50 candidates, 9 candidates exhibited an expression profile characterized by low cell specificity and/or high expression in peripheral blood mononuclear cells (PBMCs). Two 5′ UTR gene candidates, hist1h1c and rpl15, were selected for further investigation. Table 2 summarizes the initial gene screen.

TABLE 2
Summary of Endogenous Gene Bioinformatics

| | 5′ UTR | Translation | |
|---|---|---|---|
| Gene ID | Size | Rate (ksp) | Cell Type Specificity |
| hist1h1c | 43 | 147337.15 | Glandular Cells |
| | | | (eosinophils) |
| rps25 | 63 | 27023.56 | Low |
| rpl31 | 87 | 25219.48 | Low |
| rpl15 | 36 | 20242.96 | Low |
| rab11b | 96 | 19290.92 | Low |
| psma3 | 46 | 8076.35 | Low |
| rpl18a | 35 | 6465.83 | Low |
| rps29 | 30 | 3444.66 | Low |
| rbm8a | 29 | 2481.4 | Low |

### Example 2: De Novo Synthesis of 5′ UTRs

De novo synthesis of 5′ UTRs began with removal of inhibitory motifs and start codons (e.g., ATG) from the native 5′ UTR nucleotide sequence of hist1h1c and rpl15. Specifically, the 5′ terminal oligopyrimidine tract (5′ TOP) inhibitory motif, comprising repeats of cytosine and thymine/uracil, were removed. Further, a kozak consensus sequence (i.e., GCCACC) was added to the 3′ terminus of the 5′ UTR. Next, to reduce formation of secondary structures in the 5′ UTR, one or more point mutations were introduced into the 5′ UTR of hist1h1c and rpl15.

Turning to Table 3, de novo synthesis of the hist1h1c 5′ UTR is summarized. As shown in SEQ ID NO: 1 (native hist1h1c 5′ UTR), removal of a 5′ TOP was not required. Following addition of a kozak consensus sequence (SEQ ID NO: 2), one or more point mutations (i.e., a single nucleobase substitution, insertion, or deletion) were introduced into the nucleotide sequence (SEQ ID NOS: 3-15). In each sequence of Table 3, nucleobases of the kozak consensus sequence are underlined, nucleobases representing a substitution are bolded and underlined, and nucleobases representing an insertion are bolded and italicized.

Table 4 summarizes de novo synthesis of the rpl15 5′ UTR. The native rpl15 5′ UTR (SEQ ID NO: 16) required removal of a 5′ TOP, resulting in SEQ ID NO: 17. The native rpl15 5′ UTR sequence included the “GCCA” of the kozak consensus sequence, and the terminal “CC” of the kozak consensus sequence was added via nucleobase pair substitution at the 3′ terminus of the rpl15 5′ UTR (SEQ ID NO: 18). Following addition of the kozak consensus sequence, one or more point mutations were introduced into the nucleotide sequence (SEQ ID NOS: 19-23). In each sequence of Table 4, nucleobases of the kozak consensus sequence are underlined, nucleobases representing a substitution are bolded and underlined, and nucleobases representing an insertion are bolded and italicized.

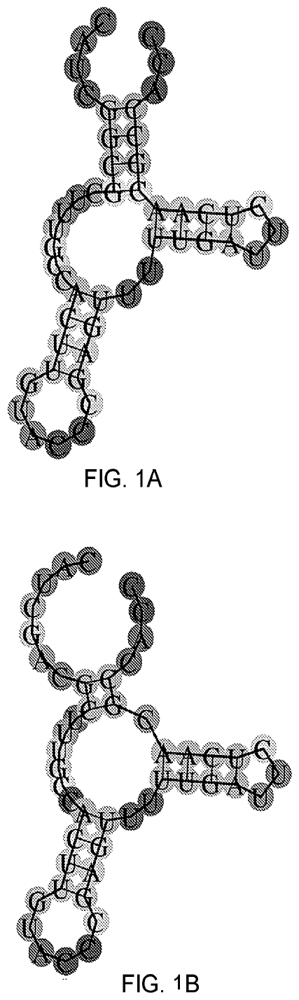

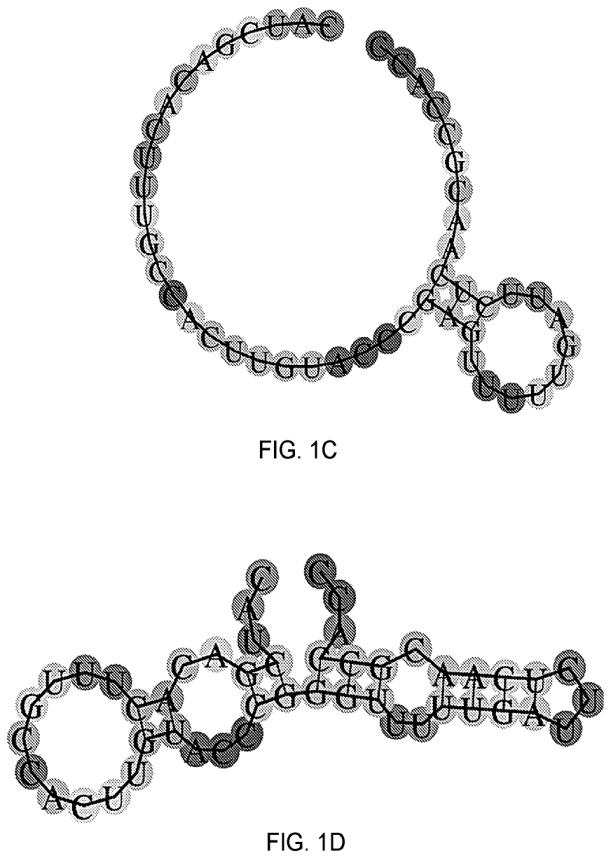

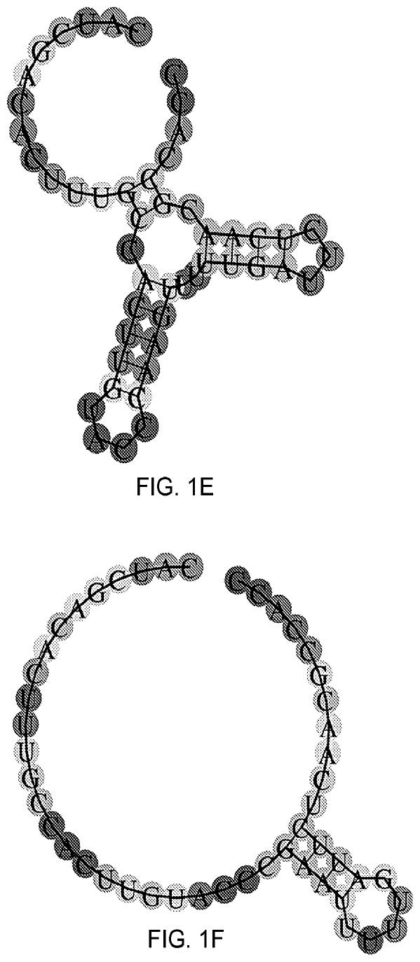

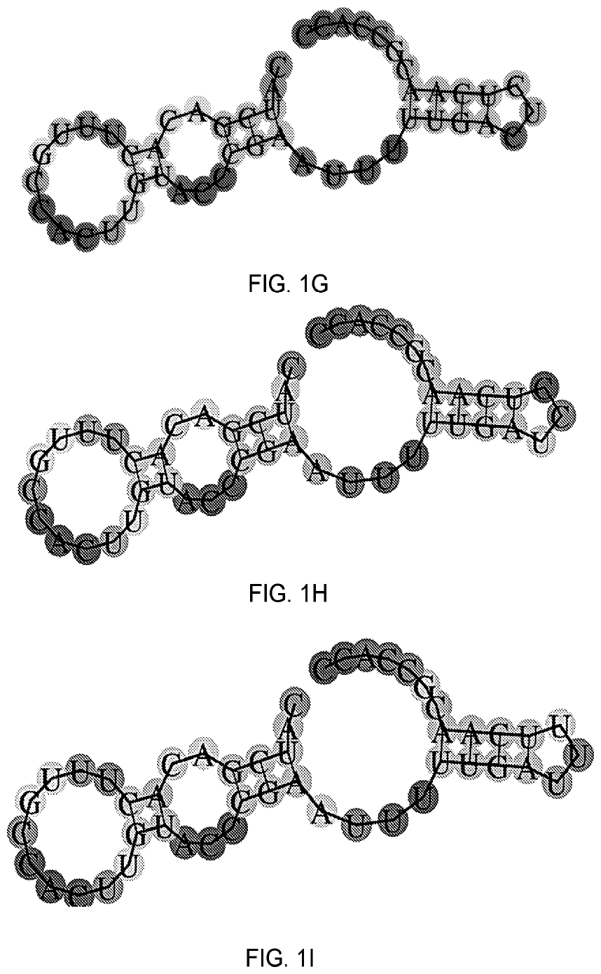

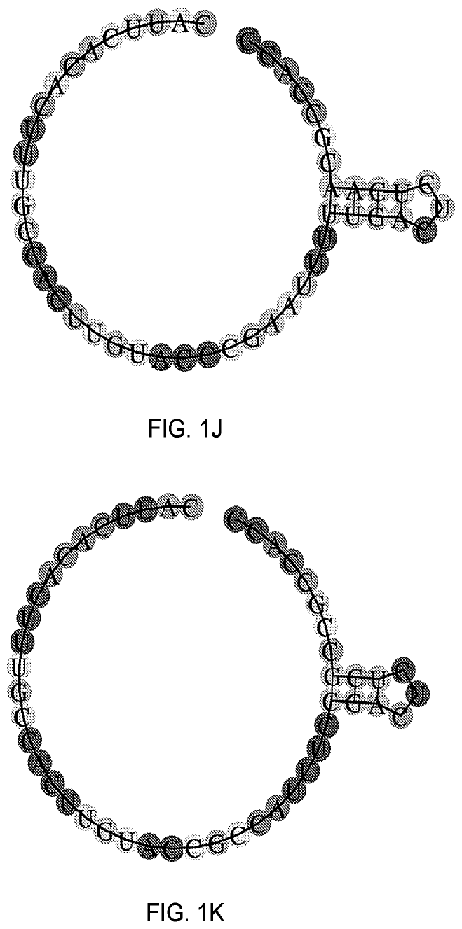

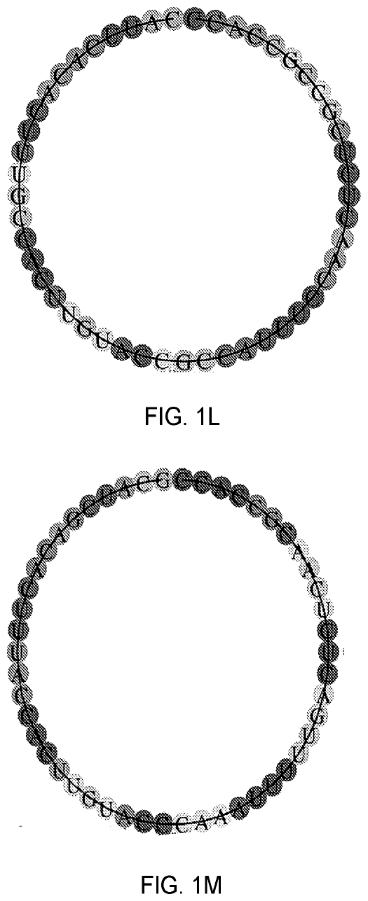

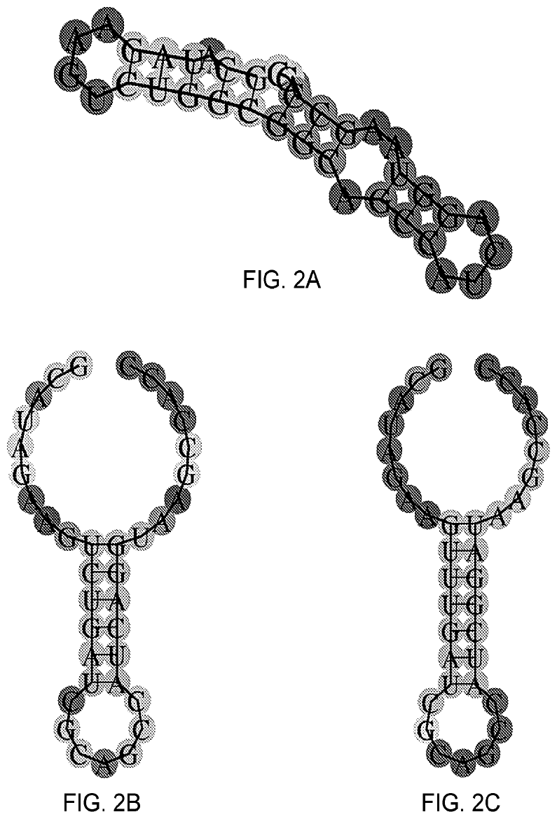

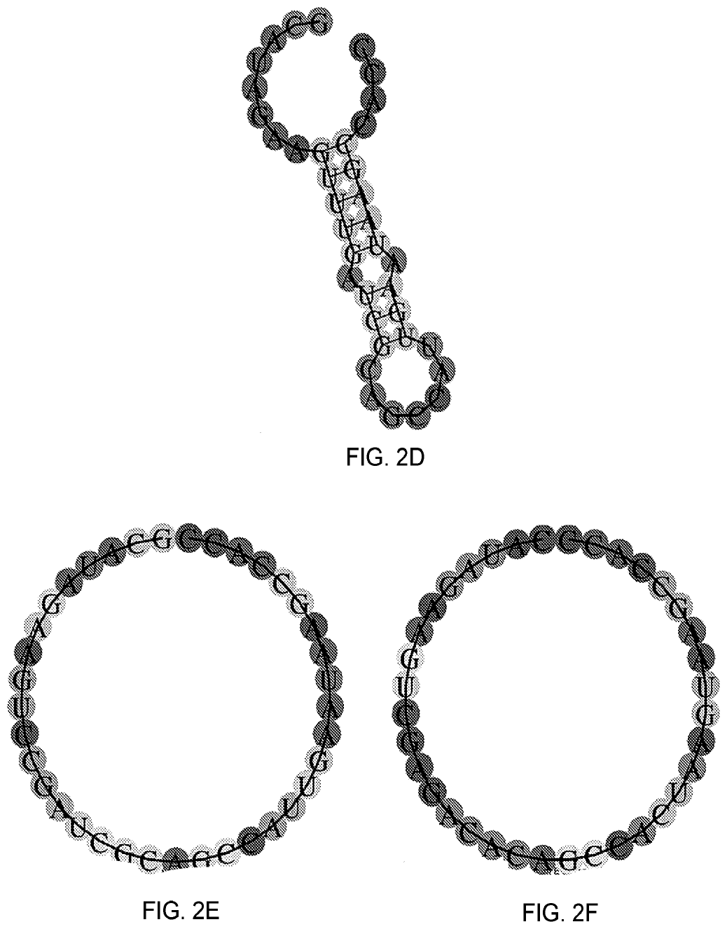

The resulting free energy of modified hist1h1c and rpl15 5′ UTRs are shown in Table 5 and Table 6, respectively. The resulting structures of modified hist1h1c and rpl15 5′ UTRs are shown FIGS. 1A-1M and FIGS. 2A-2F, respectively.

TABLE 3
De novo Synthesis of the hist1h1c 5′ UTR

| Sequence | | |
|---|---|---|
| Identifier | Sequence | SEQ ID NO |
| Native | CATCGGCGCTTTGCCACTTGTACCCGAGTTTTTGATTCTCAAC | SEQ ID NO: 1 |
| hist1h1c | | |
| 5′ UTR | | |
| hist1h1c | CATCGGCGCTTTGCCACTTGTACCCGAGTTTTTGATTCTCAACGCCACC | SEQ ID NO: 2 |
| 5′ UTR + | | |
| kozak | | |
| sequence | | |
| Mutant 1 | CATCGACGCTTTGCCACTTGTACCCGAGTTTTTGATTCTCAACGCCACC | SEQ ID NO: 3 |
| Mutant 2 | CATCGACACTTTGCCACTTGTACCCGAGTTTTTGATTCTCAACGCCACC | SEQ ID NO: 4 |
| Mutant 3 | CATCGACACTTTGCCACTTGTACCCGGGTTTTTGATTCTCAACGCCACC | SEQ ID NO: 5 |
| Mutant 4 | CATCGACACTTTGCCACTTGTACCCAAGTTTTTGATTCTCAACGCCACC | SEQ ID NO: 6 |
| Mutant 5 | CATCGACACTTTGCCACTTGTACCCGAATTTTTGATTCTCAACGCCACC | SEQ ID NO: 7 |
| Mutant 6 | CATTCACACTTTGCCACTTGTACCCGAATTTTTGACTCTCAACGCCACC | SEQ ID NO: 8 |
| Mutant 7 | CATCGACACTTTGCCACTTGTACCCGAATTTTTGATCCTCAACGCCACC | SEQ ID NO: 9 |
| Mutant 8 | CATCGACACTTTGCCACTTGTACCCGAATTTTTGATTTTCAACGCCACC | SEQ ID NO: 10 |
| Mutant 9 | CATTCACACTTTGCCACTTGTACCCGAATTTTCGACTCTCAACGCCACC | SEQ ID NO: 11 |
| Mutant 10 | CATTCACACTTTGCCACTTGTACCCGAATTTCCGACTCTCGCCGCCACC | SEQ ID NO: 12 |
| Mutant 11 | CATTCACACTTTGCCACTTGTACCGCCATTTCCGACTCTCGCCGCCACC | SEQ ID NO: 13 |
| Mutant 12 | CATTCACACTTTGCCACTTGTACCGCCATTTCCAACTCTCGCCGCCACC | SEQ ID NO: 14 |
| (“UTR3”) | | |
| Mutant 13 | GCATCGACACTTTACCACTTGTACCCAAATTTTTGACTCTCAACGCCACC | SEQ ID NO: 15 |
| (“UTR4”) | | |

TABLE 4
De novo Synthesis of the rpl15 5′ UTR

| Sequence | | |
|---|---|---|
| Identifier | Sequence | SEQ ID NO |
| Native | CCTTTCCGTCTGGCGGCAGCCATCAGGTAAGCCAAG | SEQ ID NO: 16 |
| rpl15 5′ | | |
| UTR | | |
| rpl15 5′ | GCATAGAAGTCTGGCGGCAGCCATCAGGTAAGCCAAG | SEQ ID NO: 17 |
| UTR-5′ | | |
| TOP | | |
| rpl15 5′ | GCATAGAAGTCTGGCGGCAGCCATCAGGTAAGCCACC | SEQ ID NO: 18 |
| UTR + | | |
| kozak | | |
| sequence | | |
| Mutant 14 | GCATAGAAGTCTGATCGCAGCCATCAGGTAAGCCACC | SEQ ID NO: 19 |
| Mutant 15 | GCATAGAAGTTTGATCGCAGCCATCGGATAAGCCACC | SEQ ID NO: 20 |
| Mutant 16 | GCATAGAAGTTTGATCGCAGCCATTGAATAAGCCACC | SEQ ID NO: 21 |
| Mutant 17 | GCATAGAAGTCCGATCGCAGCCATTGAATAAGCCACC | SEQ ID NO: 22 |
| (“UTR6”) | | |
| Mutant 18 | CATAGAAGTCGAGACACAGCCACTAAGTAAGCCACC | SEQ ID NO: 23 |
| (“UTR7”) | | |

TABLE 5
Free Energy of Modified hist1h1c 5′ UTRs

| Sequence | | Free Energy |
|---|---|---|
| Identifier | SEQ ID NO | (kcal/mol) |
| hist1h1c 5′ | SEQ ID NO: 2 | −8.31 |
| UTR + kozak | | |
| sequence | | |
| Mutant 1 | SEQ ID NO: 3 | −3.48 |
| Mutant 2 | SEQ ID NO: 4 | −2.93 |
| Mutant 3 | SEQ ID NO: 5 | −2.60 |
| Mutant 4 | SEQ ID NO: 6 | −3.32 |
| Mutant 5 | SEQ ID NO: 7 | −2.20 |
| Mutant 6 | SEQ ID NO: 8 | −1.16 |
| Mutant 7 | SEQ ID NO: 9 | −1.66 |
| Mutant 8 | SEQ ID NO: 10 | −1.72 |
| Mutant 9 | SEQ ID NO: 11 | −2.21 |
| Mutant 10 | SEQ ID NO: 12 | −0.39 |
| Mutant 11 | SEQ ID NO: 13 | −1.07 |
| Mutant 12 | SEQ ID NO: 14 | 0.0 |
| Mutant 13 | SEQ ID NO: 15 | 0.0 |

TABLE 6
Free Energy of Modified rpl15 5′ UTRs

| Sequence | | Free Energy |
|---|---|---|
| Identifier | SEQ ID NO | (kcal/mol) |
| rpl15 5′ UTR + | SEQ ID NO: 18 | −9.30 |
| kozak sequence | | |
| Mutant 14 | SEQ ID NO: 19 | −3.40 |
| Mutant 15 | SEQ ID NO: 20 | −2.70 |
| Mutant 16 | SEQ ID NO: 21 | −2.10 |
| Mutant 17 | SEQ ID NO: 22 | 0.0 |
| Mutant 18 | SEQ ID NO: 23 | 0.0 |

### Example 3: Luciferase Reporter Assay

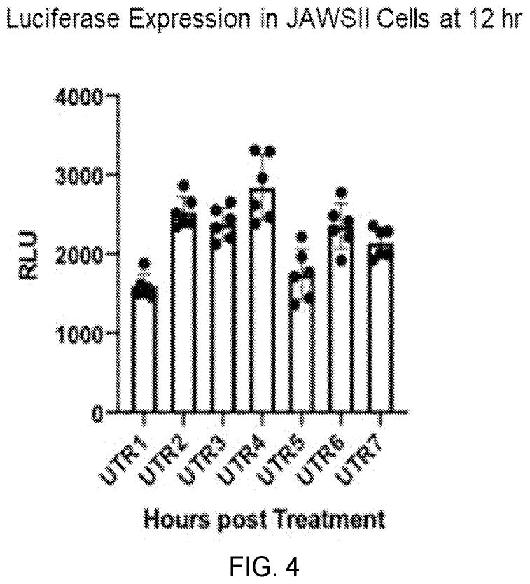

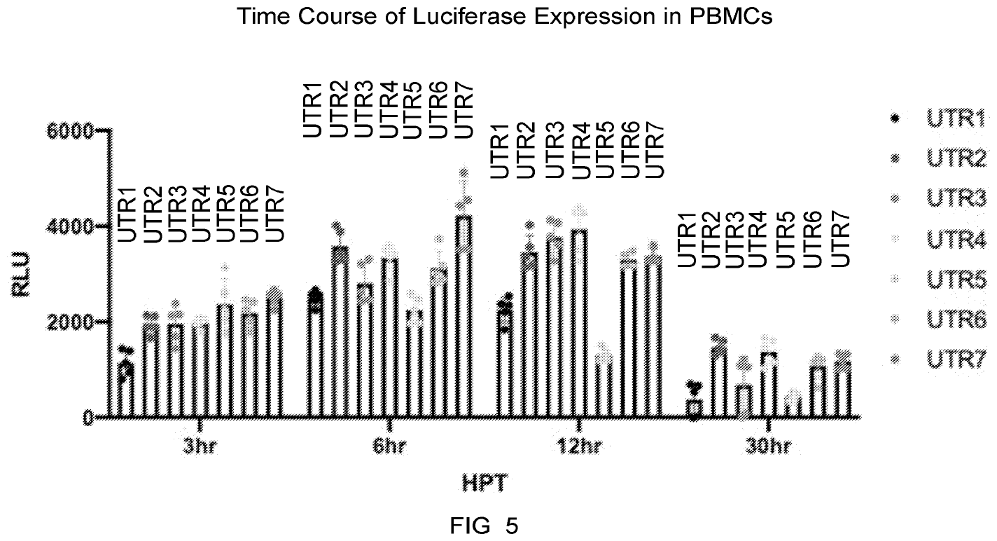

To evaluate the expression activity of UTR2-UTR7, each UTR polynucleotide was PCR-amplified and cloned into a luciferase expression vector. HEK293 cells, JAWSII (immortalized murine immature dendritic cell line), and PBMCs were independently transfected with UTR1 (a control UTR sequence), UTR2, UTR3, UTR4, UTR5, UTR6, or UTR7 and luciferase expression was measured at 3 hr, 6 hr, 12 hr, and 30 hr. FIG. 3 illustrates luciferase expression (RLU) of transfected HEK293 cells over the course of 30 hours. FIG. 4 illustrates luciferase expression of transfected JAWSII cells at 12 hours. FIG. 5 illustrates luciferase expression of transfected PBMCs over the course of 30 hours. As shown in FIG. 3, the UTRs 2, 3, 4, 6 & 7 show increased relative luminescence units as compared to the control UTR sequence, indicating an increased translations when these UTR polynucleotides are transfected in HEK293 cells. As shown in FIGS. 4 & 5, UTRs 2-7 show increased relative luminescence units as compared to the control UTR sequence, which also indicates an increased translations when these UTR polynucleotides are transfected in JAWSII cells and PBMCs.

TABLE 7
Luciferase Reporter Constructs

| UTR | Sequence Identifier | SEQ ID NO |
|---|---|---|
| UTR1 | Control UTR sequence | SEQ ID NO: 24 |
| UTR2 | Native hist1h1c 5′ UTR | SEQ ID NO: 1 |
| UTR3 | Mutant 12 | SEQ ID NO: 14 |
| UTR4 | Mutant 13 | SEQ ID NO: 15 |
| UTR5 | Native rpl15 5′ UTR | SEQ ID NO: 16 |
| UTR6 | Mutant 17 | SEQ ID NO: 22 |
| UTR7 | Mutant 18 | SEQ ID NO: 23 |

Sequence Listing

| Sequence | | |
|---|---|---|
| Identifier | Sequence | SEQ ID NO |
| Native | CATCGGCGCTTTGCCACTTGTACCCGAGTTTTTGATTCTCAAC | SEQ ID NO: 1 |
| hist1h1c 5′ | | |
| UTR | | |
| (“UTR2”) | | |
| hist1h1c 5′ | CATCGGCGCTTTGCCACTTGTACCCGAGTTTTTGATTCTCAACGCCACC | SEQ ID NO: 2 |
| UTR + | | |
| kozak | | |
| sequence | | |
| Mutant 1 | CATCGACGCTTTGCCACTTGTACCCGAGTTTTTGATTCTCAACGCCACC | SEQ ID NO: 3 |
| Mutant 2 | CATCGACACTTTGCCACTTGTACCCGAGTTTTTGATTCTCAACGCCACC | SEQ ID NO: 4 |
| Mutant 3 | CATCGACACTTTGCCACTTGTACCCGGGTTTTTGATTCTCAACGCCACC | SEQ ID NO: 5 |
| Mutant 4 | CATCGACACTTTGCCACTTGTACCCAAGTTTTTGATTCTCAACGCCACC | SEQ ID NO: 6 |
| Mutant 5 | CATCGACACTTTGCCACTTGTACCCGAATTTTTGATTCTCAACGCCACC | SEQ ID NO: 7 |
| Mutant 6 | CATTCACACTTTGCCACTTGTACCCGAATTTTTGACTCTCAACGCCACC | SEQ ID NO: 8 |
| Mutant 7 | CATCGACACTTTGCCACTTGTACCCGAATTTTTGATCCTCAACGCCACC | SEQ ID NO: 9 |
| Mutant 8 | CATCGACACTTTGCCACTTGTACCCGAATTTTTGATTTTCAACGCCACC | SEQ ID NO: 10 |
| Mutant 9 | CATTCACACTTTGCCACTTGTACCCGAATTTTCGACTCTCAACGCCACC | SEQ ID NO: 11 |
| Mutant 10 | CATTCACACTTTGCCACTTGTACCCGAATTTCCGACTCTCGCCGCCAC | SEQ ID NO: 12 |
| | C | |
| Mutant 11 | CATTCACACTTTGCCACTTGTACCGCCATTTCCGACTCTCGCCGCCAC | SEQ ID NO: 13 |
| | C | |
| Mutant 12 | CATTCACACTTTGCCACTTGTACCGCCATTTCCAACTCTCGCCGCCAC | SEQ ID NO: 14 |
| (“UTR3”) | C | |
| Mutant 13 | GCATCGACACTTTACCACTTGTACCCAAATTTTTGACTCTCAACGCCAC | SEQ ID NO: 15 |
| (“UTR4”) | C | |
| Native rpl15 | CCTTTCCGTCTGGCGGCAGCCATCAGGTAAGCCAAG | SEQ ID NO: 16 |
| 5′ UTR | | |
| (“UTR5”) | | |
| rpl15 5′ | GCATAGAAGTCTGGCGGCAGCCATCAGGTAAGCCAAG | SEQ ID NO: 17 |
| UTR-5′ | | |
| TOP | | |
| rpl15 5′ | GCATAGAAGTCTGGCGGCAGCCATCAGGTAAGCCACC | SEQ ID NO: 18 |
| UTR + | | |
| kozak | | |
| sequence | | |
| Mutant 14 | GCATAGAAGTCTGATCGCAGCCATCAGGTAAGCCACC | SEQ ID NO: 19 |
| Mutant 15 | GCATAGAAGTTTGATCGCAGCCATCGGATAAGCCACC | SEQ ID NO: 20 |
| Mutant 16 | GCATAGAAGTTTGATCGCAGCCATTGAATAAGCCACC | SEQ ID NO: 21 |
| Mutant 17 | GCATAGAAGTCCGATCGCAGCCATTGAATAAGCCACC | SEQ ID NO: 22 |
| (″UTR6”) | | |
| Mutant 18 | CATAGAAGTCGAGACACAGCCACTAAGTAAGCCACC | SEQ ID NO: 23 |
| (“UTR7”) | | |
| UTR1 | GAGAGAAAAGAAGAGTAAGAAGAAATATAAGAGCCACC | SEQ ID NO: 24 |
| (“Control”) | | |

1. A modified 5′ UTR comprising SEQ ID NO: 14, SEQ ID NO: 15, SEQ ID NO: 22, or SEQ ID NO: 23.

2. A polynucleotide sequence comprising a 5′ UTR of hist1h1c or rpl15, wherein the 5′ UTR comprises one or more point mutations that reduce formation of secondary structures in the 5′ UTR.

3. The polynucleotide sequence of claim 2, wherein the 5′ UTR is the 5′ UTR of hist1h1c, and wherein the 5′ UTR of hist1h1c lacks a start codon and comprises a kozak consensus sequence at the 3′ terminus of the 5′ UTR of hist1h1c.

4. The polynucleotide sequence of claim 3, wherein the 5′ UTR of hist1h1c comprises substitution mutations at positions 6, 8, 26, 28, and 36 from the 5′ terminus of the 5′ UTR of hist1h1c.

5. The polynucleotide sequence of claim 4, wherein the 5′ UTR of hist1h1c further comprises substitution mutations at positions 4, 5, 25, 27, 32, 33, 34, 41, and 42 from the 5′ terminus of the 5′ UTR of hist1h1c.

6. The polynucleotide sequence of claim 4, wherein the 5′ UTR of hist1h1c further comprises a substitution mutation at position 13 from the 5′ terminus of the 5′ UTR of hist1h1c.

7. The polynucleotide sequence of claim 6, further comprising an insertion mutation at the 5′ terminus of the 5′ UTR of hist1h1c.

8. The polynucleotide sequence of claim 7, wherein the insertion mutation is immediately upstream of position 1 from the 5′ terminus of the 5′ UTR of hist1h1c.

9. The polynucleotide sequence of claim 8, wherein the insertion mutation is insertion of a single nucleotide.

10. The polynucleotide sequence of claim 2, wherein the 5′ UTR is the 5′ UTR of rpl15, and wherein the 5′ UTR of rpl15 lacks a start codon and comprises a kozak consensus sequence at the 3′ terminus of the 5′ UTR of hist1h1c.

11. The polynucleotide sequence of claim 10, wherein the 5′ UTR of rpl15 comprises substitution mutations at positions 11, 13, 14, 15, 24, and 26 from the 5′ terminus of the 5′ UTR of rpl15.

12. The polynucleotide sequence of claim 11, wherein the 5′ UTR of rpl15 further comprises substitution mutations at positions 25 and 27 from the 5′ terminus of the 5′ UTR of rpl15.

13. The polynucleotide sequence of claim 11, wherein the 5′ UTR of rpl15 further comprises substitution mutations at positions 12, 16, and 23 from the 5′ terminus of the 5′ UTR of rpl15.

14. The polynucleotide sequence of claim 12, further comprising an insertion mutation at the 5′ terminus of the 5′ UTR of rpl15.

15. The polynucleotide sequence of claim 14, wherein the insertion mutation is immediately upstream of position 1 from the 5′ terminus of the 5′ UTR of rpl15.

16. The polynucleotide sequence of claim 15, wherein the insertion mutation is insertion of a single nucleotide.

17. The polynucleotide sequence of claim 2, further comprising a coding region located downstream of the 5′ UTR of hist1h1c or rpl15.

18. The polynucleotide sequence of claim 2, wherein the 5′ UTR comprises SEQ ID NO: 14, SEQ ID NO: 15, SEQ ID NO: 22, or SEQ ID NO: 23.

19. A method of increasing translation of target mRNA, the method comprising promoting translation with a modified 5′ UTR comprising SEQ ID NO: 14, SEQ ID NO: 15, SEQ ID NO: 22, or SEQ ID NO: 23, wherein the modified 5′ UTR is upstream of the target mRNA in a polynucleotide sequence.

20. A method of increasing translation of target mRNA, the method comprising promoting translation with a modified 5′ UTR of hist1h1c or rpl15, wherein the 5′ UTR comprises one or more point mutations that reduce formation of secondary structures in the 5′ UTR, wherein the modified 5′ UTR is upstream of the target mRNA in a nucleotide sequence.

21. The method of claim 20, wherein the 5′ UTR is the 5′ UTR of hist1h1c, and wherein the 5′ UTR of hist1h1c lacks a start codon and comprises a kozak consensus sequence at the 3′ terminus of the 5′ UTR of hist1h1c.

22. The method of claim 21, wherein the 5′ UTR of hist1h1c comprises substitution mutations at positions 6, 8, 26, 28, and 36 from the 5′ terminus of the 5′ UTR of hist1h1c.

23. The method of claim 22, wherein the 5′ UTR of hist1h1c further comprises substitution mutations at positions 4, 5, 25, 27, 32, 33, 34, 41, and 42 from the 5′ terminus of the 5′ UTR of hist1h1c.

24. The method of claim 22, wherein the 5′ UTR of hist1h1c further comprises a substitution mutation at position 13 from the 5′ terminus of the 5′ UTR of hist1h1c.

25. The method of claim 24, further comprising an insertion mutation at the 5′ terminus of the 5′ UTR of hist1h1c.

26. The method of claim 25, wherein the insertion mutation is immediately upstream of position 1 from the 5′ terminus of the 5′ UTR of hist1h1c.

27. The method of claim 26, wherein the insertion mutation is insertion of a single nucleotide.

28. The method of claim 20, wherein the 5′ UTR is the 5′ UTR of rpl15, and wherein the 5′ UTR of rpl15 lacks a start codon and comprises a kozak consensus sequence at the 3′ terminus of the 5′ UTR of hist1h1c.

29. The method of claim 28, wherein the 5′ UTR of rpl15 comprises substitution mutations at positions 11, 13, 14, 15, 24, and 26 from the 5′ terminus of the 5′ UTR of rpl15.

30. The method of claim 29, wherein the 5′ UTR of rpl15 further comprises substitution mutations at positions 25 and 27 from the 5′ terminus of the 5′ UTR of rpl15.

31. The method of claim 29, wherein the 5′ UTR of rpl15 further comprises substitution mutations at positions 12, 16, and 23 from the 5′ terminus of the 5′ UTR of rpl15.

32. The method of claim 30, further comprising an insertion mutation at the 5′ terminus of the 5′ UTR of rpl15.

33. The method of claim 32, wherein the insertion mutation is immediately upstream of position 1 from the 5′ terminus of the 5′ UTR of rpl15.

34. The method of claim 33, wherein the insertion mutation is insertion of a single nucleotide.
